(19) United States
(12) Patent Application Publication
(10) Pub. No.: US 2020/0164313 A1
(43) Pub. Date: May 28, 2020

(54) Connection Device For A Tubular Filter Module

(71) Applicant: FRESENIUS MEDICAL CARE DEUTSCHLAND GMBH, Bad Homburg (DE)

(72) Inventors: Thomas HEINZ, St. Wendel (DE); Torsten KELLER, St. Wendel (DE); Gerhard WIESEN, Bad Homburg (DE)

(73) Assignee: FRESENIUS MEDICAL CARE DEUTSCHLAND GMBH, Bad Homburg (DE)

(21) Appl. No.: 16/638,149

(22) PCT Filed: Aug. 14, 2018
(86) PCT No.: PCT/EP2018/071989

§ 371 Date: Feb. 11, 2020

(30) Foreign Application Priority Data
Aug. 16, 2017 (DE) 10 2017 214 263.4

(51) Int. Cl. B01D 63/06; C02F 1/44; A61M 1/16

(52) U.S. Cl. CPC B01D 63/06; C02F 1/44; A61M 1/1652; C02F 2201/004; B01D 2313/105; B01D 2313/125; B01D 2317/04; B01D 2313/06

(57) ABSTRACT

The invention relates to a connection device for receiving and connecting filter modules, in particular tubular filter modules, having at least one lateral, in particular radial, connector wherein a connecting apparatus of the connection device connects the filter module at the lateral connector and fixes it in the connection device. Further connecting apparatus of the connection device serve in the connecting of connectors arranged coaxially on the filter module, wherein the coaxial connectors are not fixed via the further connecting apparatus.

### FIELD OF THE INVENTION

The invention relates to a connection device for receiving filter modules, in particular tubular filter modules, as well as a filter module, in particular a tubular filter module, to be received in such a connection device. The invention furthermore relates to a filtration system comprising a connection device and a filter module as well as an assembly consisting of a plurality of the filtration systems. The invention furthermore relates to the use of a filter module, in particular a tubular filter module, in fluid processing processes, in particular filtration, particularly water treatment.

### BACKGROUND OF THE INVENTION

Filter modules, in particular tubular filter modules, are used in water treatment and in dialysis of patients with kidney failure. The tubular filter modules are characterized by an elongated, in particular cylindrical housing, and could be called tubes. The ends of the tubes are closed by suitable caps. Such tubular filter modules are generally designed such that the area within the tubular housing is divided into at least two flow chambers by a membrane wall; in particular, the area within the tubular housing can also be divided into at least two flow chambers by a plurality of membrane walls. In this respect, hollow fiber membrane bundles are for example used in order to separate a first and a second flow chamber in the tubular housing. The first flow chamber can thereby occupy the space between the hollow fiber membranes inside the housing, with the lumen sides of the hollow fiber membranes forming the second flow chamber. Hollow fiber membranes in tubular filter modules are generally potted in a sealing compound at the ends of the hollow fiber membranes and in the region of the end of the filter module's tubular housing such that the first flow chamber and the second flow chamber can only be in fluid communication across the membrane walls. Such filter modules are sufficiently known and utilized in the field of water treatment and the dialysis of patients with kidney failure.

A tubular filter module comprises at least two fluid inlets, whereby each flow chamber is connected to at least one fluid inlet. The influent fluid entering into the first flow chamber is thereby also referred to as the “feed.” The feed fluid is filtrated through the membrane partition wall and passes on to the second flow chamber as filtrated fluid where it can be channeled off through the second fluid inlet. The filtrated liquid is thereby also referred to as the “filtrate.”

It is furthermore known in the prior art for such modules to also comprise a third fluid inlet. Same can be arranged on the filter module so as to be fluidly connected to the first flow chamber, the feed side, or the second flow chamber, the filtrate side. This third connector can for example serve in channeling off or returning feed or filtrate. The third connector can also be provided to enable backflushing a contaminated side of the membrane. The connectors of these tubular filter modules can be connected for example via hoses and be connected to a fluid processing machine such as e.g. a dialysis machine or a water treatment system. In large filtration systems such as known for example in water treatment, the connectors are permanently connected to connecting pipes. Tubular filter modules having fluid connections attached to the respective ends of the tubular module housing are known. The fluid connections are thus arranged at the ends of the tubular housings of the filter modules in order to achieve a local separation between feed flow and filtrate side. This is to thereby prevent the feed side from cross-contaminating the filtrate side. In an upright position of the tubular filter module, the filtrate connector is usually above, the feed connector below, a third connector arranged on the side of the filter via which the retentate discharges from the filter module.

Connecting a plurality of tubular filter modules into an assembly via the fluid inlets attached to the ends of the filter modules via connecting pipes is known in the field of drinking water treatment plants. Such an interconnecting of the filter modules by means of pipes can on the one hand create a filtration cascade across multiple filter modules within an assembly. Furthermore, due to the tubular filter modules being arranged in parallel and the interconnecting of the connecting pipes, such an assembly has a self-supporting structure which lends stability and compactness to the arrangement.

The constructional compactness of such assemblies in filtration systems does, however, make it difficult to remedy defects which occur; particularly the replacement of individual filter modules in an assembly is coupled with great effort. Therefore, in order to minimize the repair expenditure, sometimes the entire assembly is replaced in the event of defects. However, this procedure is cost-intensive and, in the case of smaller filtration systems and private users, economically disadvantageous.

Furthermore, in filtration processes subject to temperature fluctuations, the described assemblies are also affected by thermally induced material expansion; i.e. particularly the longitudinal expansion of the tubular filter modules and the connecting pipes become apparent subject to the temperature change of the feed and filtrate fluids. The longitudinal expansion of the tubular filter modules thereby needs to be accommodated by the interconnected connecting pipes, which on the whole leads to material stresses and in particular also to constructional stresses within the filter module assembly. Such stresses are to be considered disadvantageous as they can lead to defects or to leakages in the fluid-conducting system of the assembly.

WO 2012/113505 A1 describes an apparatus for treating water for application in a domestic water supply. The apparatus comprises a filter head, an inlet and an outlet, a filter cup and a filter element able to be inserted into the filter cup. The filter cup is brought from a removal position into a locking position and vice versa by means of an actuating device. In the removal position, the filter element can be removed from the filter cup or a filter element can be introduced into the filter cup respectively. In the locking position, the filter cup is pressed onto the filter head so that the filter cup is pretensioned and the filter element in the filter cup connects in a sealing manner to the inlet and the outlet of the apparatus.

DE 197 27 251 A1 shows a filter apparatus and a retention system which is to enable a simple and secure connection to a hose or pipeline system. The filter apparatus thereby comprises at least two connectors, in particular four connectors, which are disposed on a side of the filter apparatus housing such that their center lines are arranged parallel. The filter apparatus is used in a water treatment unit for water filtration as utilized in the dialysis treatment of individuals suffering from kidney failure. The retaining apparatus comprises guides which can engage with counter-guide pieces on the filter apparatus. A fluidic connection of the filter apparatus is furthermore established by extendable nozzles of the retaining apparatus. The nozzles can thereby be inserted into the connectors of the filter apparatus by a lever apparatus.

US 2015/0001140 describes a filtration system for treating water which has a rotatable cover, a retaining device and a filter cartridge. The filter cartridge thereby consists of a housing having two lateral and horizontal connectors disposed on said housing. The retaining device comprises two retainers complementary to the filter connectors in order to establish a fluidic connection with the filter cartridge. The rotatable cover thereby serves as a locking apparatus and secures the position of the connectors in the complementary retainers.

The known prior art filtration systems are unsatisfactory in terms of easy handling and low-maintenance operation. The known systems do not satisfactorily solve the problem of thermal material expansion compensation. Moreover, there still further remains the need to provide a system constituting an assembly from a plurality of filter apparatus which is optimized in terms of thermal material expansion but, on the other hand, also enables replacing individual filter modules within the assembly with little effort.

### OBJECT OF THE INVENTION

In a first aspect of the invention, the object is thus that of providing a connection device for a filter module, in particular a tubular filter module, with which a fluidic connection to the filter module can be established and which enables simple replacing of a filter module, wherein the connection device is designed such that thermal material expansion does not lead to damaging material stresses.

In a further aspect of the invention, the object is that of providing a filter module, in particular a tubular filter module, which can be easily installed or replaced in a connection device, wherein the filter module is designed such that when fluidly connected to the connection device, thermal material expansion is possible without a build-up of material stresses.

In a further aspect of the invention, the object is that of providing a filtration system of a connection device and a filter module, in particular tubular filter module, which allows a thermal expansion of the filter module in the connection device and which prevents material stresses in the filtration system.

A further aspect of the invention consists of providing an assembly for a plurality of filtration systems for fluid treatment systems, in particular water treatment systems, in which filter modules, in particular tubular filter modules, are incorporated into connection device in such a manner as to enable a thermal expansion of the filter modules without the occurrence of constructional material stresses.

### SUMMARY OF THE INVENTION

In a first aspect of the invention, the object is inventively solved by a connection device in accordance with claim 1. Subclaims 2 to 8 constitute preferential embodiments of the first aspect of the invention.

In a second aspect of the invention, the object is solved by a filter module in accordance with claim 9. Subclaims 10 to 14 thereby constitute preferential implementations of the second aspect of the invention.

In a third aspect, the object is solved by a filtration system according to claim 15.

In a fourth aspect of the invention, the object is solved by an assembly according to claim 16.

In a fifth aspect of the invention, the object is solved by the use of a filter module in accordance with at least one of claims 9 to 14 in a process according to claim 17.

In a sixth aspect of the invention, the object is solved by the use of a connection device in accordance with at least one of claims 1 to 9 in a process according to claim 18.

### DETAILED DESCRIPTION OF THE INVENTION

A first aspect of the invention relates to a connection device for receiving at least one filter module, in particular a tubular filter module, which is connectable to the connection device by moving from a proximal position into a distal receiving position along a direction (E), which comprises at least one lateral connector, in particular a radial connector, having at least one first connecting apparatus with a connector piece having an inner lumen which exhibits a proximal end facing the filter module and a distal end facing the connection device in the receiving position, wherein the proximal end of the connector piece is designed to enable a connection of the connector piece to the lateral, in particular radial connector of the filter module and to at least one first and one second guide member having a respective proximal end and distal end which define a receiving area of the connecting apparatus for receiving a filter module, in particular a tubular filter module, wherein at least the first guide member exhibits a side directed toward the receiving area which has at least one guide surface running from the proximal end to the distal end of the first guide member and connecting to the proximal end of the connector piece, wherein the side of the guide member facing the guide surface directed toward the receiving area is of S-shaped configuration, particularly in cross section.

The connection device has the advantage of a filter module, in particular tubular filter module, being able to be inserted into the connecting apparatus without the lateral, in particular radial, connector needing to be tilted. In particular, the filter module with the lateral, in particular radial connector can be inserted into the receiving area of the connecting apparatus such that only the connector needs to be approximately—albeit not precisely—aligned toward the connector piece. Upon the further inserting of the tubular filter module into the receiving area of the connecting apparatus from a proximal position into a distal receiving position along a direction E, the lateral, in particular radial, connector of the tubular filter module is aligned by the at least one first guide member. During this alignment procedure, the filter module, in particular tubular filter module, is rotated about its longitudinal axis. The alignment thereby ensues from the lateral, in particular radial, connector of the tubular filter module contacting the lateral guide surfaces of the guide members which are aligned with the receiving area in the connecting direction. In the process of the tubular filter module being brought into the receiving area of the connecting apparatus, the connector is aligned along the S-shaped guide surfaces of the first guide member such that the center axis of the connector coincides with the axis of the connector piece. The tubular filter module is thus aligned such that a fluidic connection can be established between the connector and the connector piece.

The term “connection device” thereby refers to a constructional unit equipped to establish a fluidic connection to a filter module. A connection device in the sense of the present application comprises at least one or more functional elements, wherein a connecting apparatus constitutes at least one of said functional elements. The connection device can also comprise a plurality of connecting apparatus with which a fluidic connection can be established with at least one connector of the filter module in order to feed fluid into the tubular filter module or channel it out of the filter module. A connection device can also further exhibit constructional parts for the mechanical retention of the filter module. Furthermore, locking devices can also be provided with which the filter module can be fixed in the connection device or with which the fluid connection between the connection device and the connecting apparatus can be secured.

The term “tubular filter module” refers to a filter module, the housing of which can be described as a tube. The term “tube” refers to housings of circular, oval or angular cross section. Essential to the filter module being received in the receiving area of the connecting apparatus is that the filter module exhibits a cross section in one section of the lateral, in particular radial connector which is able to interact with the receiving area of the connecting apparatus. Preferentially, the tubular filter module exhibits a cylindrical housing, thus a housing having a circular cross section. However, tubular filter modules having an oval or angular housing can also be preferential as long as one section of the housing in the area of the lateral connector is formulated with a circular cross section. Such filter modules are particularly known as hollow fiber membrane filters in the prior art. In hollow fiber membrane filters, a plurality of hollow fiber membranes is arranged in a tubular cylindrical housing of a filter module. The hollow fiber membranes thereby divide the internal area of the housing into at least two flow chambers through which fluid can flow via end caps attached to the ends of the tubular housing and via the respective connectors. At least two connectors thereby serve to feed and then channel off a feed fluid and a filtrate fluid again to/from the filter module. “Tubular” is thereby to be further understood as the longitudinal extension of the housing being greater than the diameter of the filter module's cross section.

In the sense of the present application, the term “connecting apparatus” refers to a device with which a fluidic connection can be established with the connectors of the filter. The connecting apparatus comprises at least one connector piece able to be connected to the connector of the filter module. The “connector piece” thereby consists of a hose or a sleeve having a proximal and a distal end, whereby the proximal end is provided to establish a connection, in particular fluid-tight connection, to the connector of the filter module. Hoses or sleeves have an internal cavity through which the respective fluids can be led into or out of the filter module. The cavity is referred to as the “lumen” and extends from the proximal end to the distal end of the connector piece.

The term “connector” thereby refers to an inlet to the tubular filter module. The connector can be designed in the form of a sleeve and attached to the housing or to the end caps of the tubular filter module. The connector comprises at least one opening through which the fluids can pass into the first or second flow chamber of the tubular filter module or can be channeled out of said flow chambers. Preferably, the connector is of cylindrical configuration and has an inner lumen.

The connector piece can preferably consist of a flexurally rigid plastic material or a metal. The connector piece can thereby be formulated such that at least in one end section of the connector piece, the lumen exhibits an oversize in relation to the outer dimensions of the filter module connector so that a connection can be made by the connector being pushed into the connector piece. Preferably, the connector piece and the connector are of cylindrical configuration. In one alternative, however, the connector piece can also be formulated such that the lumen of the filter module connector exhibits an oversize in relation to the outer dimensions of the connector piece such that the connector piece can be inserted into the end of the connector and a connection made. A sealing of the connection can be realized by sealing elements introduced into the contact area region of the connector piece and connector. Such sealing elements are e.g. O-rings circumferentially fit to the outer surface or inner surface on the lumen side of the filter module connector. Alternatively or additionally, such O-rings can also be fit to the connector pieces on the outer or inner lumen side-surface of the connector pieces.

Additionally, the ends of the connector piece and the connector can be covered by penetrable septa, preferably slit septa, in order to prevent contamination of the connector or the connector piece. In the sense of the present application, despite the cover, the connector piece and the connector of the tubular filter module can be connected through the septa. Thus, as defined by the present application, the term “opening” also signifies openings which are covered by penetrable septa. Corresponding slit septa consist preferably of a flexible plastic material, in particular a thermoplastic elastomer material.

The connecting apparatus further comprises a first and a second guide member with a proximal end and a distal end, whereby the guide members are attached to the connecting apparatus such that a receiving area is formed between the guide members. The receiving area is provided so that a filter module, in particular a tubular filter module, can be inserted into the connecting apparatus and thereby received in the receiving area. In particular, the at least two guide members hold the filter module, in particular tubular filter module, in the receiving area. In one preferential implementation, one section of the connecting apparatus receiving area defined by the guide members is defined in a circular segment-like manner corresponding to the tubular geometry of the filter module such that the tubular filter module inserted into the connecting apparatus is enclosed by the guide members in the receiving area. That means that in an area directed toward the receiving area, the guide members have a curved form, in particular a circular-segmented form, which can attach to the housing of the tubular filter module.

The guide members further comprise at least one or more guide surfaces aligned with the receiving area of the connecting apparatus which are formed such that, on the one hand, they can enclose the filter module, in particular tubular filter module, in the receiving area but, on the other hand, are also designed such that the filter module is guided and aligned upon introduction into the receiving area of the connecting apparatus. In particular, the guide surfaces of the guide members are provided to align the connector of the tubular filter module upon introduction into the receiving area of the connecting apparatus such that the connector is directed toward the connector piece. The filter module, in particular tubular filter module, is thereby rotated about its longitudinal axis when being aligned and introduced into the receiving area. The guide surfaces of the guide members are therefore curved in further sections, particularly in circular-segmented form. The guide surfaces of the guide members are configured such that the curved sections, in particular the circular-segmented areas, are joined from the proximal end to the distal end and give the guide surface an S-shape, particularly in cross section, which connects to the proximal end of the connector piece. The “cross section” described here thereby coincides in particular with a surface extending through the receiving area and the two guide members of the connecting apparatus and within which the direction (E) can extend. The radii of the curved sections, in particular the circular-segmented sections, are thereby so complementary to one another as a function of the diameter of the filter module, in particular tubular filter module, that the connector can be aligned upon the filter module being inserted into the receiving area of the connecting apparatus. In particular, the radius of the first circular-segmented curved section connecting to the proximal end of the connector piece is selected so as to be smaller than the radius of the second circular-segmented curved section connecting to the first circular-segmented curved section. Furthermore, particularly the radius of the second circular-segmented curved section is equal to or larger than the radius of the filter module, in particular tubular filter module. In particular, the radius of the first circular-segmented curved section is less than half as large of the radius of the second circular-segmented curved section or, in alternative embodiments, the radius of the first circular-segmented curved section is smaller than the radius at the second circular-segmented curved section by an approximate factor of 0.3.

In accordance with a further embodiment, the S-shaped guide surfaces of the guide members are configured such that a linear section, to which the second circular-segmented curved section connects, is provided at the first circular-segmented curved section connecting to the proximal end of the connector piece.

In a further embodiment, the circular segments of the curved sections are configured such that the angles of the circular segments satisfy the following relationship:

$$tan α < \frac{1}{μ} \qquad Equation 1$$

whereby
α is the angle of the circular segment and
μ is the sticking coefficient of the material pair.
The material pair is formed thereby of the lateral, in particular radial connector of the filter module and the guide surface(s) of the connecting apparatus guide members. The sticking coefficient μ is a variable known to one skilled in the art. A definition can be found for example in: “Dubbel, Taschenbuch für den Maschinenbau, 22nd Ed., Springer Verlag 2007.” Adherence to Equation 1 as shown above enables particularly low-friction inserting of the filter module into the receiving area of the connecting apparatus.

The connector of the tubular filter module is thereby directed from a non-aligned position upon being inserted into the receiving area of the connecting apparatus by the lateral guide surface. The connector is thereby guided along the cross-sectionally S-shaped guide surface of the guide member and thus aligned such that in an aligned position, the opening of the filter module connector is opposite the opening at the proximal end of the connecting apparatus connector piece and can be brought into connection by the tubular filter module being inserted further.

The connection brings the connector of the filter module, in particular tubular filter module, into fluidic communication with the connection device. Preferably, the connector piece and the connector are in overlapping engagement; i.e. the connector piece is pushed into the connector of the filter module, in particular tubular filter module, when being connected or the connector of the filter module, in particular tubular filter module, is pushed into the lumen of the connector piece. The connection via connector piece and connector can be locked in placed and thus at the same time serve as a fixation for the tubular filter module in the connection device. This thereby yields the advantage of the filter module, in particular tubular filter module, being connected and secured at the lateral, in particular radial, connector via the connecting apparatus. Since the fixation is made via the connection of the radial connector, the filter module, in particular tubular filter module, can expand or contract longitudinally due to thermal expansion without disadvantageous material stresses resulting.

In a further embodiment, the second guide member likewise comprises a side directed toward the receiving area of the connecting apparatus and S-shaped in cross section having a guide surface which connects to the proximal end of the connector piece. Operation is thereby simplified by the connector of the filter module, in particular tubular filter module, being able to be guided toward the connector piece across both guide surfaces when the tubular filter module is inserted into the receiving area of the connecting apparatus. Thus the openings of the connector and the connector piece at the proximal end of the connector piece can be brought into a facing position either via the first or the second guide surface of the guide members and thus more easily and quickly connected.

In a further embodiment, the first guide member and the second guide member are cantilevered to the connector piece, preferably at the proximal end of the connector piece. Such a configuration enables an integral construction of the connecting apparatus. In particular, the connecting apparatus can be produced from plastic as a single-piece component in a single-stage injection molding process.

In a further embodiment, the S-shaped side of the connecting apparatus of the first guide member or the first guide member and the second guide member directed toward the receiving area of the filter module, in particular tubular filter module, has an upper and a lower guide surface such that a groove is arranged between the upper and the lower guide surface(s). The guide surfaces thereby run on an upper and a lower projection relative to the groove. The thus configured groove supports the guiding of the filter module, in particular tubular filter module, upon introduction into the receiving area of the connecting apparatus. It is provided for the filter module, in particular tubular filter module, which is to be brought into connection with the connecting apparatus to exhibit a circumferential bearing surface or a plurality of circumferentially arranged projections complementary to the groove, which are guided into the groove when the filter module, in particular tubular filter module, is being inserted into the receiving area of the connecting apparatus. The circumferential bearing surface thereby forms an area of the lateral, in particular radial connector of circular cross section. The inserting of the filter module, in particular tubular filter module, is thereby facilitated and is secured against the filter module tilting in the receiving area of the connecting apparatus.

In a further embodiment, the connection device is designed such that the upper projection on which the upper of the two guide surfaces of the first guide member runs is of shorter configuration at the proximal end of the guide member compared to the lower projection on which the lower guide surface runs such that the lower projection forms a platform. In an alternative embodiment, the upper projections on which the upper guide surfaces of the first guide member and the second guide member run are of shorter configuration at the proximal end compared to the lower projections on which the lower guide surfaces run such that the lower projections form platforms. The filter module, in particular tubular filter module, to be brought into the receiving area of the connecting apparatus can thus initially be set onto the platform formed by the lower projection by a circumferentially disposed bearing surface or by a plurality of circumferentially disposed projections. The circumferential bearing surface or the plurality of circumferentially arranged projections on the tubular filter module can thus be securely introduced into the groove of the guide members and the process of inserting the filter module, in particular tubular filter module, into the receiving area of the connecting apparatus made more secure and easier.

In a further embodiment, the connection device is characterized by the connecting apparatus comprising a locking mechanism. A “locking apparatus” in the sense of the present application thereby refers to mechanical locking means which prevent the filter module, in particular tubular filter module, from disengaging out of the receiving area. The locking can be by mechanical form-fit or force-fit. In one embodiment, the locking is effected by a pin inserted into a drill hole extending through the guide members of the connecting apparatus and through the circumferential projection(s) on the tubular filter module. Furthermore, pretensioned clasps, quick releases or clips can be affixed at the proximal ends of the guide members which reach from the proximal end of the first guide member to the proximal end of the second guide member and engage in the locked state of the filter module, in particular tubular filter module.

In a further embodiment, the connection device is characterized by comprising at least one further connecting apparatus for receiving at least one connector arranged coaxially to the longitudinal axis of the tubular filter module. The connecting apparatus for receiving a coaxial connector on the tubular filter module likewise comprises a connector piece provided for the connection of the coaxial connector. The connector piece has an inner lumen and can consist of a length of tubing or a sleeve. In the sense of the present application, the connecting apparatus for receiving a coaxial connector on the tubular filter module is also referred to as a coaxial connecting apparatus.

The connector piece of the coaxial connecting apparatus consists of a plastic material or a metal. The connector piece can thereby be configured such that lumen of the connector piece is oversized at least at one end section compared to the outer dimension of the coaxial connector of the filter module such that a connection can be made by the coaxial connector being pushed into the coaxial connector piece. In an alternative, the connector piece can however also be configured such that the lumen of the coaxial connector of the filter module is oversized compared to the outer dimensions of the coaxial connector piece so that the connector piece can be inserted into the end of the connector and a connection made. A sealing of the connection can be realized by sealing elements introduced into the contact area region of the connector piece and connector. Such sealing elements are e.g. O-rings circumferentially fit to the outer surface or inner surface on the lumen side of the filter module connector. Alternatively or additionally, such O-rings can also be fit to the connector pieces on the outer or inner lumen side-surface of the connector pieces.

In contrast to the connecting apparatus, which serves the connection of the lateral, in particular radial connector of the tubular filter module, the connection between the coaxial connector and coaxial connector piece is not locked by a locking apparatus. This means that the coaxial connector piece and the coaxial connector can move against each other in the connected state. In particular, such mobility can be necessary to be able to compensate longitudinal thermal expansions of the tubular filter module without material stresses.

In a second aspect, the invention relates to a filter module, in particular a tubular filter module, which can be inserted into a connection device of the type as described above. The filter module comprises a tubular housing having a first flow chamber and a second flow chamber separated from each other by at least one membrane, and further at least one first and one second connector fixed to the filter module, wherein the at least first connector is in fluid communication with the first flow chamber and the at least second connector is in fluid communication with the second flow chamber, whereby the at least first connector is laterally, preferably radially, fixed on the filter module and whereby the filter module comprises one or more circumferentially arranged projections at the height of the one first laterally disposed connector.

The filter module has the advantage that it can be placed over the circumferentially arranged bearing surface or the plurality of circumferentially disposed projections on the guide members of the connecting apparatus for connecting the lateral, in particular radial, connector in order to introduce the filter module into the connecting apparatus. The circumferential projection thereby constitutes a guiding aid in connecting the tubular filter module. Preferably, the circumferential bearing surface or the plurality of circumferential projections is utilized in inserting the filter module into the at least one groove or into both grooves of the guide members of the connecting apparatus. The groove in the at least one guide member or in the at least first and second guide member works together with the circumferential bearing surface or the circumferentially arranged projections on the filter module, in particular tubular filter module, as an improved guiding aid in the inserting of the filter module into the receiving area of the connecting apparatus. In a preferential embodiment, the circumferential bearing surface is circular or a circle segment.

In a further embodiment, the filter module, in particular tubular filter module, is characterized by the circumferentially arranged bearing surface being at the height of the central horizontal cross-sectional area of the lateral connector. The connecting apparatus can thus be of constructionally advantageous configuration.

In a further embodiment, the filter module, in particular tubular filter module, has at least three connectors arranged on the filter module, wherein the first connector is in fluid communication with the first flow chamber and the second connector and third connector are in fluid communication with the second flow chamber.

In an alternative embodiment, the first connector and the second connector are in fluid communication with the first flow chamber and the third connector is in fluid communication with the second flow chamber.

In a further embodiment, the first connector is arranged laterally, in particular radially, on the filter module, in particular tubular filter module, whereby the second and third connector are arranged coaxially to the longitudinal axis of the tubular filter module.

In a further embodiment, the filter module, in particular tubular filter module, is characterized in that at least four connectors are arranged on the filter module, wherein the first connector and the at least fourth connector are in fluid communication with the first flow chamber and the second and third connector are in fluid communication with the second flow chamber. In this connector arrangement, the first and the fourth connector are arranged laterally, in particular radially, on the filter module, whereby the second and the third connector are arranged coaxially to the longitudinal axis of the filter module. In particular, a further embodiment provides for a tubular filter module configured as such to exhibit a further circumferentially arranged bearing surface or a plurality of circumferentially arranged projections in the area of the fourth connector. A filter module of such design is also suitable as a dialysis filter.

In a further aspect, the invention relates to a filtration system consisting of a connection device in accordance with at least one implementation of the first aspect of the invention and at least one filter module, in particular a tubular filter module, in accordance with at least one embodiment of the second aspect of the invention.

In one embodiment, the filtration system is characterized by the filter module, in particular tubular filter module, being fixed in the connection device via the radial connecting apparatus and the connecting system locking device and establishing a connection to the filter module. The coaxial connecting apparatus thereby likewise establish a connection to the tubular filter module. However, the coaxial connecting apparatus are not fixed to the tubular filter module such that the coaxial connectors of the filter module are movable in the connection in the direction of the filter module's longitudinal axis. The system thus offers the advantage of the movable coaxial connections being able to compensate thermal expansions in the longitudinal direction of the filter module and the design of the system thus not needing to compensate for material stresses.

The connector pieces of the coaxial connecting apparatus can comprise displaceable sleeves which can be moved in the axial direction to the longitudinal axis of the tubular filter module. It can be provided for the displaceable sleeves to be moved by means of levers. The levers can preferentially set the sleeves into a rotational motion. It is preferential for a sloped groove to be provided in the connector piece which can convert a rotational motion of a displaceable sleeve into a translational movement in the axial direction. Such a configuration ensures the low-stress accommodating of material expansions and, at the same time, the particularly simple assembly of the filter module, particularly in respect of the coaxial connection.

In a fourth aspect, the invention relates to an assembly of a plurality of filtration systems in accordance with the third aspect of the invention which is characterized by the filtration systems with connecting channels being in fluid communication with each other. Such assemblies are particularly suitable for water treatment systems. The advantage of the inventive assembly consists of the thermal expansion of the filter modules, in particular tubular filter modules, not needing to be compensated in material expansion by structural parts of the assembly. This in particular thereby yields a safety advantage since material expansion can lead to defects in the assembly, in particular to leakages in the connection between the connecting apparatus and the connectors on the filter module.

A fifth aspect of the invention relates to the use of a filter module in accordance with one implementation of the second aspect of the invention in the processing of fluids, in particular liquids in filtration processes, particularly in water treatment.

A sixth aspect of the invention relates to the use of a connection device according to the first aspect of the invention in fluid processing procedures, in particular liquids in filtration processes, particularly in water treatment.

### DESCRIPTION OF THE INVENTION REFERENCING THE FIGURES

FIG. 1 shows a perspective exploded view of a connection device according to the invention with an inventive filter module, each in accordance with an example embodiment of the invention.

FIG. 2 shows a connecting apparatus of a connection device according to one example embodiment of the invention in a perspective side view.

FIG. 3 shows the connecting apparatus of FIG. 2 in a top view from a direction parallel the longitudinal direction of the filter module when arranged in the receiving position.

FIG. 4a shows the connecting apparatus of FIG. 2 in a further top view from a direction parallel the longitudinal direction of the filter module when being moved into the receiving position.

FIG. 4b shows a view of the connecting apparatus with the filter module of FIG. 4a in the receiving position.

FIG. 4c schematically shows the S-shaped contour of the side of the guide member of the connecting apparatus of FIGS. 1 to 4b with the guide surface.

FIG. 5 shows a perspective side view of an inventive assembly according to one example embodiment comprising a plurality of filtration systems and the at least one inventive connection device according to one example embodiment as well as inventive filter modules according to one example embodiment.

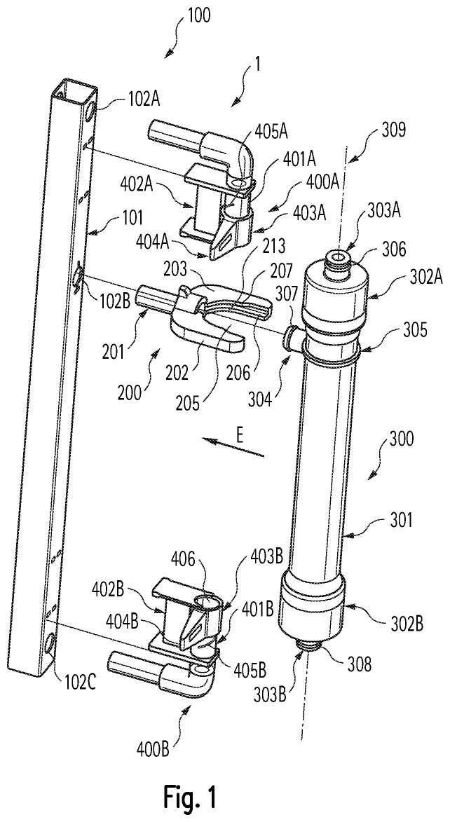

FIG. 1 is a depiction of an inventive embodiment of a filtration system 1 in the form of an exploded view. The filtration system consists of a connection device 100, a tubular filter module 300, a connecting apparatus 200 for connecting to a radial connector 304 of the filter module 300 and two connecting apparatus 400A, 400B for connecting to coaxial connectors 303A, 303B of the filter module. The connecting apparatus 200 comprises a connector piece 201 via which fluids can be fed to or drained from a flow chamber of the filter module through the radial connector 304 of the filter module 300. The connecting apparatus 200, 400A, 400B are thereby part of the connection device 100. The connection device 100 serves the accommodating and the connecting of the filter module 300 through its movement along direction (E) from a proximal position, which can be represented by the user, into a receiving position which, starting from the proximal position, is distally situated.

Guide members 202, 203 are further shown on the connecting apparatus 200, connected to the connector piece 201 of the connecting apparatus 200. The guide members exhibit guide surfaces 207, 213 directed toward the receiving area 205, of which only the guide surfaces on guide member 203 are visible in FIG. 1. FIG. 1 furthermore shows a tubular filter module 300 which can be brought into the receiving area 205 in order to connect the connector 304 to the connector piece 201 and establish a fluidic connection between the tubular filter module 300 and the connection device 100. The tubular filter module exhibits a cylindrical housing 301, its outer diameter dimensioned such that it can be received in the receiving area 205 of the connecting apparatus 200. The tubular filter module 300 exhibits further connectors 303A, 303B arranged coaxially to the longitudinal axis 309 of the tubular filter module 300. The coaxial connectors 303A, 303B are thereby arranged at the ends of the tubular filter module and connect at the end caps 302A, 302B of the tubular filter module. O-rings 306, 307, 308 are circumferentially applied to the connectors at the radial connector 304 and the coaxially arranged connectors 303A, 303B, these being provided to seal the connectors vis-à-vis the connector pieces 201, 401A, 401B of the connecting apparatus 200, 400A, 400B.

In the representation as shown, the connection device 100 comprises the supporting structure 101 which receives the connecting apparatus 400A, 200, 400B via drill holes 102A, 102B, 102C. By inserting the filter module into the connecting apparatus 200, which connects the radial connector 302, and attaching the connecting apparatus 400A, 400B, which connects the coaxial connectors 303A, 303B, the connection device 100 and the tubular filter module 300 together constitute the filtration system 1. The filter module is thereby fixed in the connection device by the connecting apparatus 200 at the radial connector 304 of the filter module and a locking device not shown in the figure. The connecting apparatus 400A, 400B, which connect the coaxial connectors of the filter module 300, are thereby configured such that the connection is not fixed.

The connecting apparatus 400A, 400B comprise the connector pieces 401A, 401B for the connecting of the coaxial connectors 303A, 303B. The connector piece 401A can also be configured as or respectively comprise a sight glass in order to enable tightness testing of the membranes by way of a bubble test. The connector pieces 401A, 401B furthermore comprise displaceable sleeves 403A, 403B which can displace axially to the longitudinal axis. In the depicted embodiment, the displaceable sleeves can be moved by levers 404A, 404B. The levers 404A, 404B serve to set the sleeves 403a, 403B into a rotational motion. A sloped groove 405A, 405B converts the rotational motion into a translational motion such that the sleeve is moved in the axial direction. In accordance with the embodiment shown in FIG. 1, the outer diameter of the coaxial connectors 303A, 303B are dimensioned such that they can be received in the lumen 406 of the displaceable sleeves. The lumen 406 of the displaceable sleeves 403A, 403B is only visible on one of the connecting apparatus 400B shown in the perspective representation of FIG. 1.

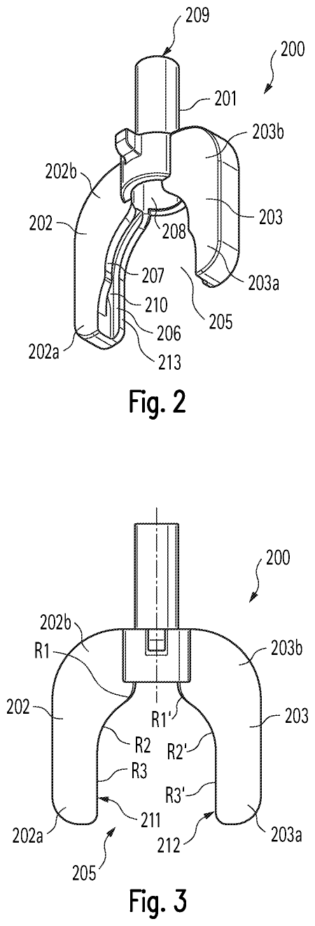

FIG. 2 shows a perspective schematic representation of an embodiment of the connecting apparatus 200 as provided for the connecting of the radial connector of the filter module. FIG. 2 thereby shows a detailed depiction of the connecting apparatus 200 from FIG. 1. The connecting apparatus 200 comprises a connector piece 201 exhibiting a proximal end 208 having an opening to the lumen of the connector piece and a distal end 209. Furthermore shown are the guide members 202, 203 attaching at the proximal end of the connector piece 208. The guide members 202, 203 define a receiving area 205 in the connecting apparatus 200 which is provided for receiving a tubular filter module. An upper 207 and a lower 213 guide surface can be seen in the FIG. 2 perspective representation. The guide surfaces are arranged so as to extend on projections opposite of the groove 210 situated between the guide surfaces. The groove 210 is thereby provided to receive the circumferential bearing surface 305 of the filter module, in particular tubular filter module 300, depicted in FIG. 1 when the filter module 300 is introduced into the receiving area 205 of the connecting apparatus 200. The guide surfaces 207, 213 shown in the FIG. 2 depiction are designed such that the projection on which the upper guide surface 207 runs is of shorter configuration than the projection on which the lower guide surface 213 runs. In thus doing, the projection on which the lower guide surface runs forms a platform 206. This platform serves in bearing the tubular filter module by the circumferential bearing surface before the tubular filter module is inserted further into the receiving area 205 of the connecting apparatus. At the same time, the platform serves in bringing the circumferential bearing surface 305 of the filter module 300 into a position for the circular bearing surface 305 to be introduced into the groove 210. The guide surfaces 207, 213 and the groove 210 are thereby configured on the guide members 202, 203 such that they extend from a proximal end 202a, 203a of the guide members 202, 203 to a distal end 202b, 203b of the guide members. In particular, the guide surfaces 213, 207 and the groove 210 are arranged so as to connect to the proximal end 208 of the connector piece 201 by the distal ends 202b, 203b of the guide members 202, 203.

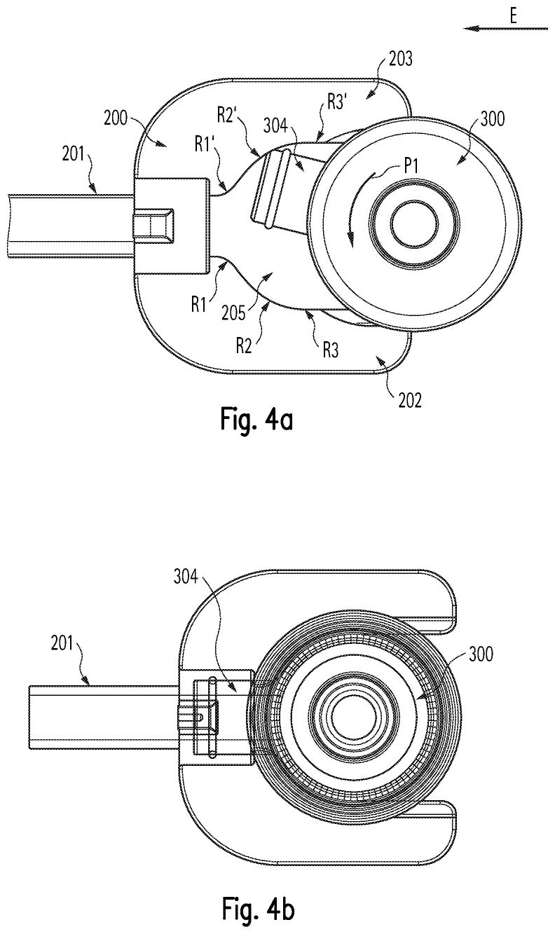

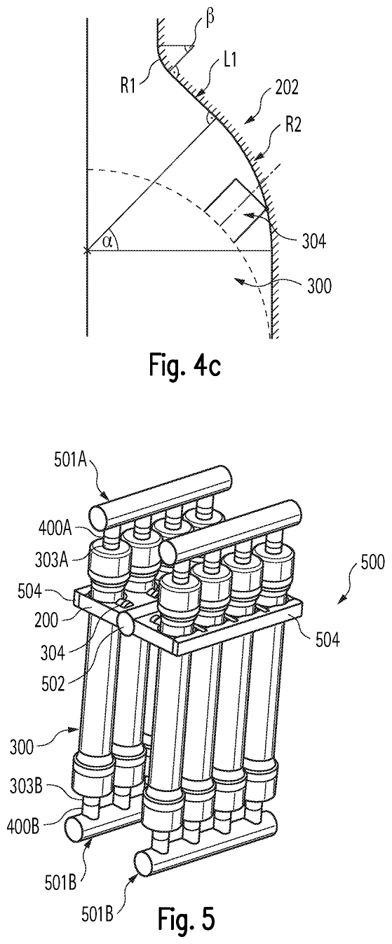

FIG. 3 shows a top view of a connecting apparatus 200 showing the respective S-shaped side 211 of the guide members 202, 203 directed toward the receiving area 205. The S-shape can on the one hand particularly be seen here in a cross section along direction E—preferably perpendicular to the longitudinal direction of a filter module—through the guide members (see FIGS. 4a, 4b), although preferably can also be seen in the top view from a direction parallel to the longitudinal direction of a filter module. Technically, it is the surface which the filter module contacts during insertion along direction E which exhibits the S-shape. The S-shape results from two sequentially assembled curved sections (R1, R1′, R2, R2′) on the guide members 202, 203 which are directed toward the receiving area 205 at the distal end 202b, 203b. A third section (R3, R3′) constitutes a straight section of the guide members at the proximal end 202a, 203a. The clearance in the receiving area 205 in the area of the straight section (R3, R3′) is thereby substantially equal to the outer diameter of the filter module 300 intended to be introduced into the receiving area. The outer diameter of the filter module 300 and the receiving area clearance are thereby adapted such that the filter module can be inserted into the receiving area without tilting. The receiving area clearance thereby exhibits a slight oversize in relation to the outer diameter of the filter module. The curved sections (R1, R1′, R2, R2′) can be described by curvature radii. The radii of curvature are thereby dimensioned such that they align the radial connector 304 of the tubular filter module 300 when the filter module is being introduced into the receiving area 205 of the connecting apparatus to the proximal end 208 of the connector piece so that the connector piece 201 and the radial connector 304 can be brought into connection.

FIG. 4a shows the connecting apparatus 200 of FIG. 2 in a further top view from a direction parallel the longitudinal direction of the filter module 300 as it is moved into the receiving position from a proximal position. Shown is a position of the filter module 300 in which the radial connector 304 is in a non-aligned position in the receiving area 205. A “non-aligned position” thereby means that the radial connector 304 is not aligned toward the connector piece 201 of the connecting apparatus 200 and thus not connectable. The guide surfaces of the guide members are not shown in FIG. 4a. The FIG. 4a representation shows the S-shaped side of the guide members 202, 203 directed toward the receiving area 205 on which the guide surfaces run. FIG. 4a furthermore shows the circular-segmented curved sections (R1, R1′, R2, R2′). The circular-segmented curved sections, the diameter of the filter module 300 and the clearance of the receiving area 205 in the straight section (R3, R3′) of the guide members 202, 203 are configured such that the radial connector 304 bears against one of the lateral guide surfaces of the guide members 202, 203 not shown in FIG. 4a when the filter module 300 is being introduced into the receiving area 205. The radial connector of the filter module 300 is then only ever in contact with one of the two guide members 202, 203 upon introduction into the receiving area 205. In the present FIG. 4a, the aligning of the radial connector is supported only by the circular-segmented curved sections of a guide member 203. By the tubular filter module 300 being inserted further into the receiving area 205 of the connecting apparatus 200, the radial connector 304 is guided along the guide surfaces (not shown in FIG. 4a) and deflected by the circular-segmented curved sections (R1′, R2′) of the guide member 203 such that the filter module rotates about its longitudinal axis in the receiving area. The rotational direction of the rotation is indicated in FIG. 4a by the P1 arrow. The tubular filter module is thereby brought into a second position in which the radial connector 304 is aligned with the connector piece and a connection is made between the radial connector 304 and the connector piece 201.

FIG. 4b shows a top view of the connecting apparatus 200 with the filter module 300 of FIG. 4a in the receiving position, in which the filter module 300 is situated in a distal receiving position and the connector 304 is in an aligned position. In the “aligned position,” the radial connector 304 of the filter module and the connector piece 201 are in a connected state. FIG. 4b thereby shows the end position of the filter module 300 in the connecting apparatus 200 which the filter module 300 assumes by being rotated further in the arrow direction (P1) from the non-aligned position of FIG. 4a and pushed into the receiving area 205.

In FIGS. 4a/b, the radius of circular-segmented curved section R1 is smaller than the radius of circular-segmented curved section R2. The radius of circular-segmented curved section R2 is equal to or greater than the radius of the tubular filter module. In particular, the radius of circular-segmented curved section R1 is less than half as large as the radius of curved section R2 or, in alternative embodiments, the radius at curved section R1 is smaller by a factor of 0.3 than the radius at curved section R2. This special design to the radii ratios enables the optimal configuring of the desired rotational motion.

In one specific implementation, the radii of the circular-segmented curved sections have the following values:

Radius at section R1: 18.63 mm
Radius at section R2: 57.15 mm
Outer diameter of tubular filter module: 57.15 mm

The outer diameter of the filter module and the clearance in sections R3, R3′ are substantially the same size, although a clearance fit of the filter module in the receiving area is provided.

FIG. 4c shows a schematic cross-sectional representation of a part of a filter module 300 in a connecting apparatus 200 in a non-aligned position. FIG. 4c shows the S-shaped contour of the side of a guide member 202 in contact with the radial connector 304 of the filter module. The guide surfaces of the guide member 202 are not shown in FIG. 4c. The contour of the schematically depicted guide member 202 directed toward the receiving area 205 is to be equated in the schematically depicted embodiment with the geometry of the guide surfaces. An embodiment of the connecting apparatus is furthermore shown in the depicted FIG. 4c representation in which the S-shaped side of the guide member 202 directed toward the receiving area exhibits a linear section (L1) connecting to the first circular-segmented curved section (R1) and the second circular-segmented curved section (R2). Analogously, the guide surfaces of both guide members can also exhibit a linear section (L1). Furthermore shown are the α and β angles of the circular segments in curved sections R1 and R2. The FIG. 4c representation clarifies the correlation, according to which the following condition is observed for aligning the filter module 300 from a non-aligned position as per FIG. 4a into an aligned position as per FIG. 4b:

$$tan α < \frac{1}{μ}$$

wherein:
α is the angle of the circular segment in R2 and
μ is the sticking coefficient of the material pair.
The analogous relationship applies to the circular segment R1 with the β angle. The material pair is formed from the radial connector 304 and the guide surface of the guide member not shown in FIG. 4c. Preferably, the radial connector 304 and the guide surface consist of plastic such as e.g. a polypropylene material or of polyvinyl chloride. Other materials can however also be used such as, for example, metal. The same or different materials can be used for the respective components. The angle of the circular segment α and the linear section L1 can be accordingly adapted for low-friction inserting of the filter module into the receiving area as a function of the sticking coefficient μ.

FIG. 5 shows one embodiment of an inventive assembly 500 consisting of a plurality of filtration systems. Tubular filter modules 300 are shown which are introduced into connecting apparatus 200 for the connection of the radial connector 304 and which are connected to the coaxial connectors 303A and 303B via further connecting apparatus 400A and 400B. The connecting apparatus 200, 400A, 400B create a fluidic connection with connecting tubes 501A, 501B, 502 with which fluids can be fed to or channeled out of the tubular filter modules. In particular, the connecting tubes can be connected to a hydraulic system of a water treatment system. A locking device 504 fixes the tubular filter modules in the assembly connecting apparatus at the radial connector 304. The connecting apparatus 400A, 400B do not, however, fix the coaxial connectors 303A, 303B in the connecting apparatus. The coaxial connectors can therefore move in the connecting apparatus 400A, 400B in the longitudinal direction of the tubular filter modules upon thermal material expansion. Thus, thermal material expansion only transmits non-damaging material stresses, or even no material stresses at all, to the connecting tubes 501A, 501B, 502.

1. A connection device for receiving at least one filter module which is connectable to the connection device by moving from a proximal position into a distal receiving position along a direction, and having at least one lateral connector, said connection device comprising
at least one first connecting apparatus
having a connector piece with an inner lumen exhibiting a proximal end facing the filter module and a distal end facing the connection device in the receiving position, wherein the proximal end of the connector piece is designed to enable a connection of the connector piece to the lateral connector, of the filter module, and
having at least one first and one second guide member exhibiting a respective proximal end and distal end which define a receiving area of the connecting apparatus for receiving a filter module, wherein at least the first guide member exhibits a side directed toward the receiving area which has at least one guide surface running from the proximal end to the distal end of the first guide member and connecting to the proximal end of the connector piece,
wherein
the side of the guide member facing the guide surface directed toward the receiving area is of S-shaped configuration in cross section.

2. The connection device according to claim 1, wherein the second guide member exhibits a side directed toward the receiving area of the connecting apparatus which has at least one guide surface running from the proximal end to the distal end of the second guide member and connecting to the proximal end of the connector piece, wherein the side of the guide member comprising the guide surface directed toward the receiving area is of S-shaped configuration in cross section.

3. The connection device according to claim 1, wherein the first guide member and the second guide member are cantilevered to the connector piece.

4. The connection device according to claim 1, wherein the S-shaped side of the at least one guide member directed toward the receiving area of the connecting apparatus or the S-shaped sides directed toward the receiving area of the at least first guide member and the second guide member comprise upper and lower guide surfaces and a groove extending along the guide surfaces between same is arranged such that the guide surfaces run on upper and lower projections relative to the groove.

5. The connection device according to claim 4, wherein the upper projection on which the upper of the two guide surfaces of the first guide member runs is of shorter configuration at the proximal end of the guide member compared to the lower projection on which the lower guide surface runs such that the lower projection forms a platform, or wherein the upper projections on which the upper of the two guide surfaces of the first guide member and the second guide member run are of shorter configuration at the proximal ends of the guide members compared to the lower projections on which the lower guide surfaces run such that the lower projections form platforms.

6. The connection device according to claim 1, wherein the connection device comprises a locking mechanism in order to fix the filter module in the receiving area of the connecting apparatus.

7. The connection device according to claim 1, wherein the connecting apparatus comprises at least one further connecting apparatus for receiving at least one connector arranged coaxially to the longitudinal axis of the filter module.

8. The connection device according to claim 7, wherein the connecting apparatus for receiving at least one connector arranged coaxially on the filter enables a movable supporting of the connector in the connecting apparatus.

9. A filter module to be received in a connection device in accordance with claim 1, comprising a tubular housing, a first flow chamber and a second flow chamber which are separated from each other by at least one membrane, at least one first and at least one second connector attached to the filter module, wherein the at least one first connector is in fluid communication with the first flow chamber and the at least one second connector is in fluid communication with the second flow chamber, wherein the one first connector is laterally fixed on the filter module,
wherein
the filter module comprises a circumferentially arranged bearing surface or a plurality of circumferentially arranged projections at the height of the first laterally arranged connector.

10. The filter module according to claim 9, wherein the circumferentially arranged bearing surface is an annular projection.

11. The filter module according to claim 9, wherein the circumferentially arranged bearing surface is at the height of the central horizontal cross-sectional area of the lateral connector.

12. The filter module according to claim 9, wherein at least three connectors are arranged on the filter module, wherein the first connector is in fluid communication with the first flow chamber and the second connector and third connector are in fluid communication with the second flow chamber, or wherein the first connector and the second connector are in fluid communication with the first flow chamber and the third connector is in fluid communication with the second flow chamber.

13. The filter module according to claim 9, wherein the at least second connector and/or the at least third connector is/are arranged coaxially to the longitudinal axis of the filter module.

14. The filter module according to claim 9, wherein the filter module comprises a plurality of hollow fiber membranes which separate at least one first flow chamber from at least one second flow chamber.

15. A filtration system comprising at least one connection device according to claim 1 and at least one filter module comprising a tubular housing, a first flow chamber and a second flow chamber which are separated from each other by at least one membrane, at least one first and at least one second connector attached to the filter module, wherein the at least one first connector is in fluid communication with the first flow chamber and the at least one second connector is in fluid communication with the second flow chamber, wherein the one first connector is laterally fixed on the filter module,
wherein
the filter module comprises a circumferentially arranged bearing surface or a plurality of circumferentially arranged projections at the height of the first laterally arranged connector.

16. An assembly comprising a plurality of filtration systems in accordance with claim 15 and connecting channels arranged between said filtration systems.

17. A method to filter a liquid comprising utilizing the filter module in accordance with claim 9.

18. A method to filter a liquid comprising utilizing the connection device in accordance with claim 1.
